(19) United States
(12) Patent Application Publication
(10) Pub. No.: US 2010/0068264 A1
(43) Pub. Date: Mar. 18, 2010

(54) NIOSOME-HYDROGEL DRUG DELIVERY SYSTEM

(75) Inventors: Norma Alcantar, Tampa, FL (US); Eva C. Williams, Tampa, FL (US); Ryan Toomey, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/622,693

(22) Filed: Nov. 20, 2009

Related U.S. Application Data
(63) Continuation-in-part of application No. 11/737,271, filed on Apr. 19, 2007.
(60) Provisional application No. 60/807,122, filed on Jul. 12, 2006.

(51) Int. Cl. A61K 9/127

(52) U.S. Cl. 424/450

(57) ABSTRACT

Localized drug delivery systems are effective means to administer therapeutic concentrations and controlled release of drugs. A delivery system consisting of non-ionic surfactant vesicles (niosomes) packaged within a biodegradable, temperature and pH sensitive hydrogel network was developed. Drug behaviors were modeled using a fluorescent dye with similar physical properties as therapeutic drugs for cancer. The niosomes were embedded into a biodegradable hydrogel providing a stable niosome environment. A cross linked chitosan was used as the hydrogel, which is a liquid at room temperature, and gels inside the body. Depending on the conditions to which individual niosomes are exposed, the release rate can be controlled to last from 24 hours to more than 3 months.

### CROSS REFERENCE TO RELATED APPLICATION

This application is a continuation-in-part of U.S. application Ser. No. 11/737,271, entitled “Niosome-Hydrogel Drug Delivery, filed Apr. 19, 2007, which claims priority to U.S. Provisional Patent Application No. 60/807,122 filed Jul. 12, 2006 entitled “Drug-Encapsulated Niosomes Embedded in Biodegradable Polymer Hydrogels for Use in Drug Delivery Applications”, the contents of which are herein incorporated by reference.

### FIELD OF INVENTION

This invention relates to drug delivery systems. Specifically, the invention relates to controlling the release rate of a therapeutic drug using nanoparticle vesicles embedded in hydrogel networks.

### BACKGROUND OF THE INVENTION

Various drug delivery systems have been developed to lessen the toxicity and improve the efficacy of drugs. Anti-neoplastic and anti-cancer agents are of particular concern, due to the high cellular toxicity inherent in many of these drugs.

Ovarian Cancer is the fourth leading cause of death by cancer in women (ca. 15280 deaths in 2007 in the United States) (Greenlee R T, Hill-Harmon M B, Murray T, Thun M. Cancer Statistics, 2005. CA Cancer J Clin 2005; 51:15-36; Jemal, A., A. Thomas, T. Murray, and M. Thun, Cancer Statistics, 2002. CA: A Cancer Journal for Clinicians, 2002. 52: p. 23-47), the leading cause of death from gynecologic malignancies and the second most commonly diagnosed gynecologic malignancy. Ovarian cancer detection in the early stages is difficult because most women show little to no symptoms until the cancer has progressed to an advanced stage and become difficult to treat, with the relative survival rate at a low 46% (Greenlee R T, et al. Cancer Statistics, 2005. CA Cancer J Clin 2005; 51:15-36; Schwartz P E, Taylor K J. Is early detection of ovarian cancer possible? Ann Med 1995; 27:519-28). Surgery is the first step in the treatment and is frequently necessary for diagnosis. Chemotherapy is typically administered after surgery to treat any residual tumors. The traditional chemotherapeutic administration techniques include intravenous (IV) injection of the drugs directly into the blood stream (Armstrong, D. K., et al., Intraperitoneal cisplatin and paclitaxel in ovarian cancer. New England Journal of Medicine, 2006. 354(1): p. 34-43; Markman, M., et al., Combination Intraperitoneal Chemotherapy with Cisplatin, Cytarabine, and Doxorubicin for Refractory Ovarian-Carcinoma and Other Malignancies Principally Confined to the Peritoneal-Cavity. J Clin Oncol, 1984. 2(12): p. 1321-1326). This technique has been used in the past years and has been successful in containing the spread of tumors and hence treating many types of cancer. Since it is not localized it exposes the whole body to the chemotherapy drugs. Hence, apart from destroying tumor cells they also attack normal healthy cells (Markman, M., et al., Combination Intraperitoneal Chemotherapy with Cisplatin, Cytarabine, and Doxorubicin for Refractory Ovarian-Carcinoma and Other Malignancies Principally Confined to the Peritoneal-Cavity. J Clin Oncol, 1984. 2(12): p. 1321-1326) resulting in extensive side effects.

Drug delivery systems, such as physical encapsulation or liposomes containing neutral or zwitterionic lipids, are used to improve drug administration. The lipids in liposomes arrange themselves into bilayers and entrap one (unilameliar) or more (oligo- or multilamellar) spaces. The spaces between the bilayers of the lipids are usually filled with water. The liposome-encapsulated drugs are entrapped in the internal aqueous space. Conventional liposomes, which rely upon the internal entrapment of the drug, often have difficulty entrapping a high concentration of a drug, as the efficiency depends upon the volume of fluid outside of the liposomes and circumscribed within the internal aqueous vesicular space. During long-term storage, drugs entrapped within liposomes leak from the internal aqueous space into the surrounding milieu, causing the drug to be lost from its desired intra-liposomal location.

Other techniques to prevent cancer recurrence include intraperitonial chemotherapy. This is a localized technique as drugs are delivered directly into the intraperitonial cavity (Armstrong, D. K., et al., Intraperitoneal cisplatin and paclitaxel in ovarian cancer. New Eng J of Med, 2006. 354(1): p. 34-43) using a catheter, but also presents challenges. Tumors in the abdominal cavity are exposed to higher concentrations of drug for longer periods of time, resulting in increased hematologic, metabolic and neurologic toxicity (Armstrong, D. K., et al., Intraperitoneal cisplatin and paclitaxel in ovarian cancer. New Eng J of Med, 2006. 354(1): p. 34-43; Markman, M., et al., Combination Intraperitoneal Chemotherapy with Cisplatin, Cytarabine, and Doxorubicin for Refractory Ovarian-Carcinoma and Other Malignancies Principally Confined to the Peritoneal-Cavity. Journal of Clinical Oncology, 1984. 2(12): p. 1321-1326; Hamilton, C. A. and J. S. Berek, Intraperitoneal chemotherapy for ovarian cancer. Curr Opinion in Oncol, 2006. 18(5): p. 507-515). Also, the catheters may become plugged over time (Hamilton, C. A. and J. S. Berek, Intraperitoneal chemotherapy for ovarian cancer. Curr Opinion in Oncol, 2006. 18(5): p. 507-515) leading to infections and other complications. Moreover, this technique is available only to select patients with minimal residual tumors (Gore, M., A. du Bois, and I. Vergote, Intraperitoneal chemotherapy in ovarian cancer remains experimental. J Clin Oncol, 2006. 24(28): p. 4528-4530).

Accordingly, what is needed is an improved drug delivery system that allows progressive release of drug over a prolonged period of time.

### SUMMARY OF THE INVENTION

This invention incorporates encapsulating a therapeutic drug in a nanoparticle vesicle that is subsequently embedded into a hydrogel network and allows for two-mode control over the release rate of the drug. The invention improves on how medication is administered to patients and reduces adverse side effects associated with over-dosage. This invention is designed for applications at physiological temperature and pH conditions. This invention will be of great interest for use in drug delivery devices, particularly for the treatment of diseases such as cancer. The invention will allow for decreased side effects and increased survival time in patients. Of particular note, this invention permits treating cancer tumors locally as well as cancers that have been partially removed through surgical procedures and that have produced a body cavities. This invention opens the door to other technological applications that require controlled release of chemical substances.

The invention is also not limited to drug delivery for brain tumor patients. It may be modified and applicable to any illness in which a site-specific delivery of active ingredient is needed. Other cancers such as breast cancer are prime candidates for the invention because the invention directly targets the infectious site. This invention is also useful in other commercial applications that require the controlled release of a product such as in pesticides or water treatment applications. Alternative embodiments of this invention are useful in the controlled release of chemical substances for engineering applications such as battery packaging and antifouling agents.

An embodiment of this invention addresses the problem of on-site brain tumor treatment by providing a controlled release of drugs to malignant cancer cells. Benefits to the patient include offering more effective techniques of eliminating cancer cells that may still be present after surgery and thus providing better health conditions following treatment. For example, the drug delivery device may be used by a surgeon after major brain surgery, or to be implanted in the tumor cavity of cancer patients. The invention could be used in applications where radiation or other harsh treatment methods are not a plausible option and injection-diffusion delivery is the best alternative.

In some embodiments of the invention, the delivery system is used in conjunction with anti-cancer therapies, such as surgical resection. After surgery the tumor resection sites are inhomogeneous (Markman, M., et al., Combination Intraperitoneal Chemotherapy with Cisplatin, Cytarabine, and Doxorubicin for Refractory Ovarian-Carcinoma and Other Malignancies Principally Confined to the Peritoneal-Cavity. J of Clin Oncol, 1984. 2(12): p. 1321-1326), making it difficult for drugs to reach each and every part of the tumor cavity. The delivery system is particularly useful here since in the matrix is liquid at room temperature, allowing it to be injected into the tumor cavity. As the matrix reaches body temperature, the matrix gels, thereby taking the shape of the cavity and ensuring uniform exposure of drugs to every part of the residual tumor.

The treatment of malignant cancer cells after major ovarian cancer resection or brain surgery is one such area that could benefit from the application of nanostructured materials. Traditional cancer treatments, such as chemotherapy, are not practical options in this situation due to the sensitivity and care that must be taken when dealing with matters of the brain. Applications for this technology include brain cancer patients who have been diagnosed with glioblastoma multiforme (glioma). These patients are in need of alternative chemotherapy treatment that is less harsh than traditional chemotherapy techniques used for other types of cancer. The sensitivity and delicate nature of the human brain makes this invention desirable to brain cancer patients and an excellent potential alternative to treatment.

The invention includes a drug delivery medium comprising at least one niosome embedded in a polymer hydrogel. The niosome is comprised of a hydrophobic bilayer defining an interior hydrophilic space with at least one hydrophobic drug integrated into the hydrophobic bilayer and at least one hydrophilic drug encapsulated in the interior hydrophilic space. The hydrogel properties are preselected in accordance with a desired release rate for the niosomes and may include biodegradability, cross-link density, pH-sensitivity and temperature sensitivity. In addition to hydrogel properties, niosome constituents are preselected in accordance with a desired release rate for the encapsulated drug and may include surfactants such as, without limiting the scope of the invention, crown ether amphiphiles bearing a steroidal moiety, 1,2-dialkyl glycerol polyoxyethylene ether, hexadecyl poly-5-oxyethylene ether, hexadecyl poly-5-oxyethylene ether (C₁₆EO₅); octadecyl poly-5-oxyethylene ether (C₁₈EO₅); hexadecyl diglycerol ether (C₁₆G₂); sorbitan monopalmitate (Span 40) and sorbitan monostearate (Span 60), Solulan™ C24 (poly-24-oxyethylene cholesteryl ether), polysorbate 20, Span detergents, Brij detergents, such as Brij-35, and polyoxyethylene, and polysorbates. Other components of the niosomes include, cholesterols, and, optionally, negative charged molecules such as dicetyl phosphates, cetyl sulphate, phosphatidic acid, phosphatidyl serine, oleic acid, palmitic acid. The niosome also optionally comprises at least one cholesterol.

The niosome optionally includes at least one negative charged molecule, such as polyoxyethylene (61), sorbitan monostearate, cetyl sulphate, phosphatidic acid, phosphatidyl serine, oleic acid, palmitic acid, and dicetyl phosphates. In specific embodiments of the drug delivery system, the niosome comprises sorbitan monostearate, at least one cholesterol, and dicetyl phosphate at a ratio of 1:1:0.1. The properties of any niosomal system are determined by hydration of the niosome, the nature of the drug, the composition of the membrane, addition of kinetic energy and/or size reduction (Uchebgu, et al.; Non-ionic surfactant based vesicles (niosomes) in drug delivery. Int J Pharm, 1998 Oct. 15; 172(1):33-70). These variables must be carefully controlled during the design and manufacture of a niosomal drug delivery system.

In specific embodiments, the drug delivery medium is a smart-packaging system, incorporating a double control mechanism that allows for the maximum determination of the release rate. Specifically, without limiting the invention to any theories, the medium release rate is controllable via adjustments to the niosomes, the hydrogel, or both. The term ‘smart-packaging’ is used due to the fact that it is responsive to external stimuli, such as, to temperature. In some embodiments, the system is a clear liquid at 25° C. (room temperature), but becomes an opaque non-flowing gel as the temperature is raised to 37° C. (body temperature).

The hydrogel of the drug delivery medium is optionally chitosan. In some embodiments, the chitosan has a molecular weight within the range of 19,000 Daltons and 31,000 Daltons. Further, the polymer hydrogel may also include a cross-linking molecule, such as B-glycerophosphate. Other useful cross-linking materials include polyols and/or polyoses. Nonlimiting examples include glycerol, pentaerythritol, ethylene glycol, glycerin, castor oil, sucrose polyethylene glycol, polypropylene glycol, poly(tetramethylene ether) glycol, trehalose, glycogen, cellulose, chitin, amylose, and amylopectin glycogen. In specific embodiments, the β-glycerophosphate is added at a ratio of chitosan to B-glycerophosphate of 4.0:1. The hydrogel also optionally comprise a plurality of biodegradable polymer hydrogel layers, where the niosomes are embedded in the hydrogel layers. In such embodiments, the adjoining hydrogel layers have distinct properties. In embodiments employing a plurality of biodegradable polymer hydrogel layers, the layers may optionally be sandwiched together thereby forming a gradient network to package vesicles in varying microenvironments to tune release rates from an identical population of vesicles wherein adjoining hydrogel layers have distinct properties. The distinct properties of the hydrogel layers may include cross-link density, pH-sensitivity and temperature sensitivity.

Also disclosed is a method of preparing a drug delivery medium, by combining at least one surfactant, at least one cholesterol, and at least one hydrophobic drug together. The surfactant and cholesterol are dissolved in a solvent to form a solution, and the solution optionally agitated at 60° C. until the solids dissolve. The solvent is evaporated to form a thin film. In some embodiments, the solution is evaporated by passing N₂ gas over the solution. The thin film is then hydrated with a hydrophilic drug. In some embodiments, the surfactant is selected crown ether amphiphiles bearing a steroidal moiety, 1,2-dialkyl glycerol polyoxyethylene ether, hexadecyl poly-5-oxyethylene ether, hexadecyl poly-5-oxyethylene ether (C₁₆EO₅); octadecyl poly-5-oxyethylene ether (C₁₈EO₅); hexadecyl diglycerol ether (C₁₆G₂); sorbitan monopalmitate, sorbitan monostearate, poly-24-oxyethylene cholesteryl ether, polysorbate 20, Span detergents, Brij detergents, polyoxyethylene, and polysorbates.

The niosomes optionally also include at least one negative charged molecules, such as polyoxyethylene (61), sorbitan monostearate, cetyl sulphate, phosphatidic acid, phosphatidyl serine, oleic acid, palmitic acid, and dicetyl phosphates. In specific embodiments, the noisome is composed of sorbitan monostearate, cholesterol and dicetyl phosphate combined in the ratio 1:1:(0.1). The niosomes are optionally extruded. Unincorporated hydrophilic drug may be separated from the niosomes by ultracentrifugation.

In addition to forming niosomes for the drug delivery medium, the niosomes are optionally added to a hydrogel, such as chitosan. Additionally, B-glycerophosphate may be added to the chitosan solution, for example at a ratio of B-glycerophosphate to chitosan of 4.0:1. In specific embodiments, the ratio of niosome to chitosan ratios ranges from (0.15):1 to (0.45):1.

### BRIEF DESCRIPTION OF THE DRAWINGS

For a fuller understanding of the invention, reference should be made to the following detailed description, taken in connection with the accompanying drawings, in which:

FIGS. 1(a) and (b) are chemical structural diagrams depicting exemplary vesicle forming crown ethers. (A) A cholestanyl derivative and (B) a cholesteryl derivative.

FIG. 2 is a cross-section illustration of a non-ionic surfactant vesicle/niosome, showing the membrane bilayer and internal compartment.

FIG. 3 is a graph showing the size distribution of niosomes as a function of the encapsulated dye. The linear trend of the plot assists in predicting the size of niosomes for a given dye concentration.

FIG. 4 is a graph showing dye release from niosomes when exposed to deionized water. Difference in the concentration of ions between niosomes and deionized water led to an increase in the osmotic pressure resulting in an uncontrolled rupture of the niosomes and hence a higher release as compared to PBS, depicted in FIG. 5.

FIG. 5 is a graph showing dye release from niosomes when exposed to PBS.

FIG. 6 is a graph showing the percentage of dye released when packaged within varying ratios of niosome to chitosan.

FIG. 7 is a bar graph depicting the percentage of dye released from the chitosan-niosome drug delivery system after 55 days. A niosome:chitosan ratio of (0.35):1 was seen to have the finest controlled release.

FIG. 8 is a graph showing a comparison of release rate from niosomes using ultracentrifugation (UC) and gel exclusion chromatography (GEC) to remove excess dye or drug.

FIGS. 9(a) and (b) are graphs showing the model fit for dye diffusion using: (A) Water; and (B) PBS medium. Solid line represents the non-linear curve fit. Symbols—Closed and Open represent the experimental data points at different times when water and PBS were used as the release medium respectively.

FIG. 10 is a graph showing the release rate of the dye with respect to the molecular weight of chitosan.

FIG. 11 is a graph showing changes in the release rate with respect to the cross-link density of the chitosan network.

FIGS. 12(a) through (c) are images of hydrogel, taken from Surface Forces Apparatus experiments. (A) Fringes in air without chitosan, control distance (mica-mica contact); (B) a compressed chitosan layer, showing a 5.6-5.8 nm thick compressed layer; (C) a chitosan loading experiment, showing more than 200 nm distance in between the surfaces.

FIG. 13 is a data plot demonstrating linear release of molecules retained in niosomes according to an embodiment of the invention.

FIGS. 14(a) and (b) are cross sections of the niosomes used in one embodiment of the invention. (A) A close up view of the delivery system containing two types of niosomes in the same solution hydrophobic drug Paclitaxel within the bilayer or hydrophilic drug Carboplatin in the hydrophilic core. (B) The two types of niosomes are mixed in solution, creating a heterogeneous mixture of niosomes, containing either hydrophobic drug or hydrophilic drug.

FIGS. 15(a) and (b) are cross sections of the niosomes used in one embodiment of the invention. (A) A close up view of the delivery system with a hydrophobic drug Paclitaxel and hydrophilic drug Carboplatin encapsulated in the same niosome (cocktail niosomal formulation). The hydrophobic drug is encapsulated within the bilayer while the hydrophilic drug is encapsulated in the in the hydrophilic core. (B) The niosomes are suspended in solution, creating a homogeneous mixture of niosomes, containing both hydrophobic drug and hydrophilic drug in each niosome.

FIGS. 16(a) through (c) are TEM images showing (A, B) cross-linked chitosan at varying densities and (C) cross-linked chitosan with embedded niosomes.

### DETAILED DESCRIPTION OF THE PREFERRED EMBODIMENT

Hydrogel structures that embed niosomes have particular utility for three types of uses. The first use is passive packaging for vesicles of different sizes, cargo, or membrane composition. This allows for the embedment of a drug or drugs that come in varying sizes and shapes. This also allows for embedment of multiple drugs in the same hydrogel network. The second use takes advantage of the gradient network to package the niosomes in controlled microenvironments. This characteristic allows one to manipulate the release rate of the drug by altering chemical and physical properties such as cross-link density of the hydrogel. Additionally, a plurality of layers of hydrogel may be used in the invention. In such embodiments each hydrogel layer may be formed independently using a preselected amount of cross-linking polymer. The niosomes are mixed with the hydrogel before gellation and the layer gelled. Subsequent layers may be added with different amounts of cross-linking or other characteristics. The cross-linked characteristic makes hydrogels resistant to dissolution and ideal for encapsulating smaller particles such as niosomes.

### Example 1

### Niosome Preparation

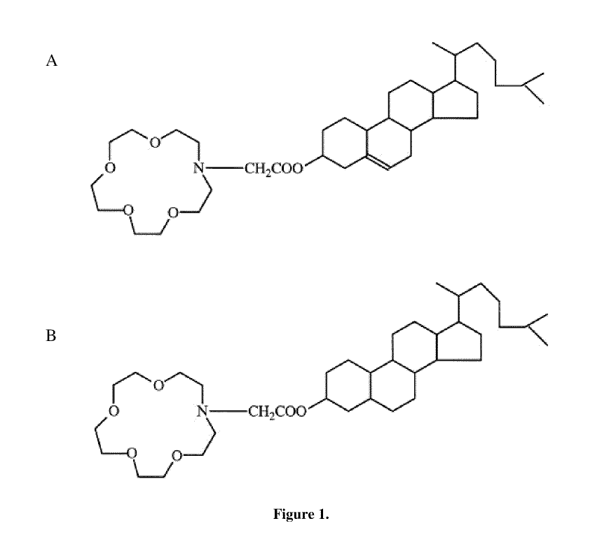

The advantage of using niosomes as opposed to liposomes is that the synthetic niosomes have shown to be more chemically stable as vesicles, they are easier to transport and store, they are less expensive, and they have been shown to increase the blood brain barrier permeability. It is composed of synthetic amphiphilic surfactants and cholesterol that make up a bilayer membrane and is able to entrap hydrophilic solutions in the aqueous core and hydrophobic solutions in the non-polar membrane. Exemplary surfactants include, without limiting the scope of the invention, crown ether amphiphiles bearing a steroidal moiety, 1,2-dialkyl glycerol polyoxyethylene ether, hexadecyl poly-5-oxyethylene ether, hexadecyl poly-5-oxyethylene ether (C₁₆EO₅); octadecyl poly-5-oxyethylene ether (C₁₈EO₅); hexadecyl diglycerol ether (C₁₆G₂); sorbitan monopalmitate (Span 40) and sorbitan monostearate (Span 60), Solulan™ C24 (poly-24-oxyethylene cholesteryl ether), polysorbate 20, Span detergents, Brij detergents, such as Brij-35, and polyoxyethylene. Examples of crown ethers are illustrated in FIGS. 1(a) and (b), and are known in the art (Echegoyen, L. E., et al., Aggregation of steroidal lariat ethers—the 1st example of non-ionic liposomes (niosomes) formed from neutral crown ether compounds. J Chem. Soc Chem Commun, 1988. 12, 836-837; Montserrat, K., et al., Light-induced charge injection in functional crown ether vesicles. J Am Chem Soc, 1980. 102, 5527-5529; Darwish, I. A., Uchegbu, I. F., The evaluation of crown ether based niosomes as cation containing and cationsensitive drug delivery systems. Int'l J Pharm, 1997. 159, 207-213; Uchegbu, I. F., Duncan, R., Niosomes containing N-(2-hydroxypropyl)methacrylamide copolymer-doxorubicin (PK1): effect of method of preparation and choice of surfactant on niosome characteristics and a preliminary study of body distribution. Int'l J Pharm, 1997. 155, 7-17). Exemplary solvents involved in noisome formation may include glycerol, oil, water, and combinations thereof.

Cholesterol provides stability to the vesicles by decreasing their permeability and enhancing solute retention (Uchegbu, I. F. and Florence, A. T., Non-ionic Surfactant Vesicles (Niosomes): Physical and Pharmaceutical Chemistry. Advances in Colloidal and Interface Science, 1995. 58: p. 1-55; Nasseri, B, Effect of cholesterol and temperature in the elastic properties of niosomal membranes. International Journal of Pharmaceuticals, 2005. 300: p. 95-101). More permeable membrane (cholesterol free) entrap a lower amount of the drug, decreasing the encapsulation efficiency (Uchegbu, I. F. and Vyas, S. P., Non-ionic Surfactant Based Vesicles (Niosomes) in Drug Delivery. International Journal of Pharmaceutics, 1998. 172: p. 33-70; Uchegbu, I. F. and Florence, A. T., Non-ionic Surfactant Vesicles (Niosomes): Physical and Pharmaceutical Chemistry. Advances in Colloidal and Interface Science, 1995. 58: p. 1-55).

In addition, negative charged molecules may be added to the bilayer-producing compounds, such as dicetyl phosphate, cetyl sulphate, phosphatidic acid, phosphatidyl serine, oleic acid, palmitic acid. Dicetyl Phosphate is used to provide electrostatic stabilization to the vesicles which prevents their aggregation (Uchegbu, I. F. and Vyas, S. P., Non-ionic Surfactant Based Vesicles (Niosomes) in Drug Delivery. International Journal of Pharmaceutics, 1998. 172: p. 33-70; Manosroi, A; et al., Characterization of vesicles prepared with various non-ionic surfactants mixed with cholesterol. Colloids and Surfaces: Biointerfaces, 2008. 30: p. 129-138). The ability of the surfactant to form a vesicle depends on two factors, the Hydrophobic Lipophilic Balance (HLB) and the Critical Packing Parameter (CPP). The HLB is calculated using

HLB=20×Mh/M (1)

Where Mh is the molecular mass of the hydrophilic portion of the surfactant, and M is the molecular mass of the whole niosome, giving a result on an arbitrary scale of 0 to 20. For the surfactant sorbitan monostearate, an HLB number between 4 and 8 was found to be compatible with vesicle formation (Uchegbu, I. F. and Vyas, S. P., Non-ionic Surfactant Based Vesicles (Niosomes) in Drug Delivery. Int'l J of Pharm, 1998. 172: p. 33-70).

The CPP is a dimensionless number that predicts the ability of the amphiphile to form aggregates. Israelachvili (Israelachvili, J. N., Intermolecular and Surface Forces: With Applications to Colloidal and Biological Systems. 1985, Orlando: Academic Press) reports that a CPP value of 0.5-1.0 predicts that the amphiphile will form a vesicle. CPP is calculated using

CPP=υ/l_{c}aₒ (2)

where υ=hydrocarbon chain volume, l_{c}=critical hydrophobic chain length (the length above which the chain fluidity of the hydrocarbon may no longer exist), and aₒ=area of hydrophilic head (Uchegbu, I. F. and Florence, A. T., Non-ionic Surfactant Vesicles (Niosomes): Physical and Pharmaceutical Chemistry. Advances in Colloidal and Interface Science, 1995. 58: p. 1-55).

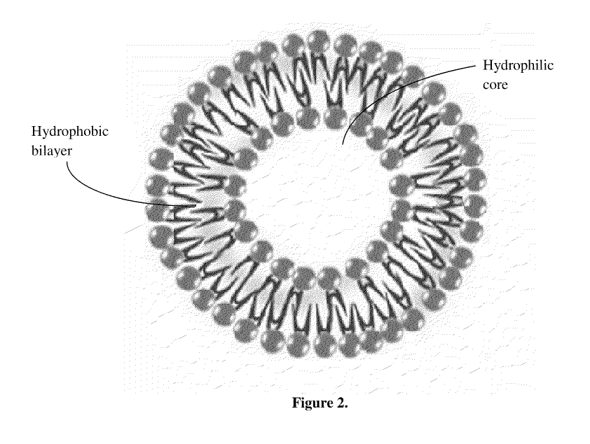

The Niosomes were formed from the self-assembly of non-ionic amphiphiles in aqueous media resulting in closed bilayer structures (Uchegbu, I. F. and Vyas, S. P., Non-ionic Surfactant Based Vesicles (Niosomes) in Drug Delivery. Int'l J of Pharm, 1998. 172: p. 33-70), seen in FIG. 2. The assembly into bilayers is rarely spontaneous and usually involves some input of energy such as physical agitation or heat. The result is an assembly in which hydrophobic parts of the molecule are shielded from the aqueous solvent and the hydrophilic head groups enjoy maximum contact with same (Uchegbu, I. F. and Vyas, S. P., Non-ionic Surfactant Based Vesicles (Niosomes) in Drug Delivery. Int'l J of Pharm, 1998. 172: p. 33-70; Israelachvili, J. N., Intermolecular and Surface Forces: With Applications to Colloidal and Biological Systems. 1985, Orlando: Academic Press).

Niosomes were prepared by combining surfactant, cholesterol, and, optionally, negative charged molecules. For example, Span 60, cholesterol and dicetyl phosphate were combined in the ratio 1:1:(0.1) in a glass tube. 3 ml of chloroform or other solvent was added to it and agitated over a 60° C. bath until all the solids dissolved. Three milliliters of this solution was transferred to a clean and dry round bottom flask and attached to a buchï rotary evaporator. Any reduction in the volume of solution was compensated for by adding an equal amount of solvent. N₂ gas was passed through the flask while rotating till the chloroform evaporates, forming uniform thin layer on the flask. N₂ gas flow was continued for at least 10 min after all of the chloroform has evaporated.

The flask is left upturned in a fume hood for at least 12 hrs, to overnight. The thin film was then hydrated using a hydrophilic drug or carboxyfluorescein dye. Four milliliters of the solution was taken and the flask then attached to a rotary evaporator while the bath is maintained at 60° C. for 1 hr or until all the film dissolved. Size reduction was performed using an extruder maintained at 60° C. for 12 cycles, leaving a solution containing niosomes with the encapsulated drug or dye and the unencapsulted dye which is separated using an ultracentrifuge at 60000 rpm for 40 minutes.

To generate niosomes containing two drugs, a hydrophobic and hydrophilic drug, the thin film is prepared by combining surfactant, cholesterol, a hydrophobic drug, and, optionally, negative charged molecules. As an example only, niosomes were created with Span 60, cholesterol, dicetyl phosphate and the Paclitaxel. Hydration is performed using a hydrophilic drug, such as Carboplatin. During the hydration process the hydrophobic parts of the system are shielded away from the hydrating solution whereas the hydrophilic parts share absolute contact with the same. Because the hydrophobic drugs are packaged in different regions of the niosome than the hydrophilic drugs, the drugs do not come in contact at any point until the niosomes are delivered to the target site. Therefore, any kind of hydrophobic and hydrophilic drug can be used for encapsulation. The combination hydrophobic-hydrophilic-drug niosome can be employed to include a wide variety of hydrophobic and hydrophilic drugs, such as Triciribine and Carboplatin.

All the mixture of drugs that have been used in ‘combination chemotherapy’ can be used for encapsulation in the drug delivery system. In a first method to produce niosomes utilizing thin films described above, the surfactant Span 60, cholesterol and dicetyl phosphate, hydrating them with the dye, constricting their size by extrusion and removal of the free dye by ultracentrifugation. In a second method all the steps till the hydration were the same after which they were sonicated for 15 min. The free dye was removed using Gel Exclusion Chromatography. The niosomes prepared by the two methods were compared for stability, encapsulation efficiency and dye release rate. The size distribution of the niosomes was determined by Dynamic Light Scattering and Transmission Electron Microscopy.

The size distribution of niosomes was found to be function of the encapsulated dye, provided by

Average Size (nm)=749.25 (nm)+23.16 (nm/mM)*┌Dve Concentration (mM)┐

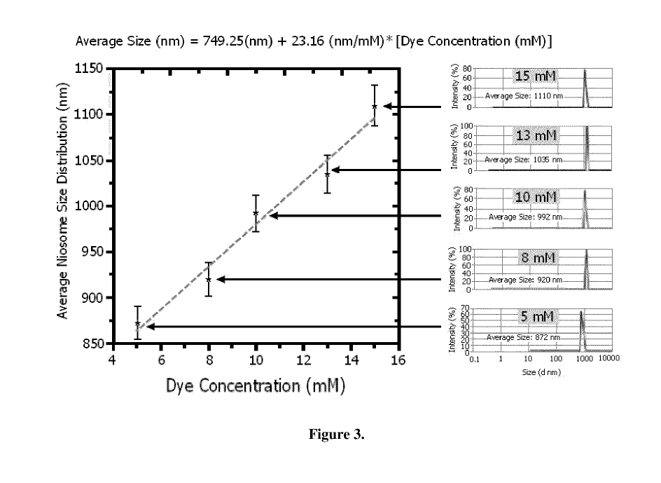

The linear trend of the plot assists in predicting the size of niosomes for a given dye or drug concentration. A range of dye concentrations from 5 millimolar (mM) to 15 millimolar (mM) were tested for niosome size and release rate, as seen in FIG. 3.

### Example 2

### Characterization of Niosomes with Integrated Drug/Dye

Niosomes were prepared by thin film hydration of the surfactant Sorbitan monostearate (Span 60), cholesterol and dicetyl phosphate in a solvent chloroform, using thin film hydration method (Uchegbu, I. F. and Vyas, S. P., Non-ionic Surfactant Based Vesicles (Niosomes) in Drug Delivery. Int'l J of Pharm, 1998. 172: p. 33-70; Uchegbu, I. F. and Florence, A. T., Non-ionic Surfactant Vesicles (Niosomes): Physical and Pharmaceutical Chemistry. Advances in Colloidal and Interface Science, 1995. 58: p. 1-55; Manosroi, A; et al.; Characterization of vesicles prepared with various non-ionic surfactants mixed with cholesterol. Colloids and Surfaces: Biointerfaces, 2008. 30: p. 129-138). Two different protocols were followed for their synthesis, as described above.

Niosomes prepared with the first method showed a linear behavior in their size distribution as the concentration of the dye was increased from 5 millimolar to 15 millimolar, as seen in FIG. 3. It was found that niosomes prepared by the second method showed no such relationship between the size distribution and the dye concentration.

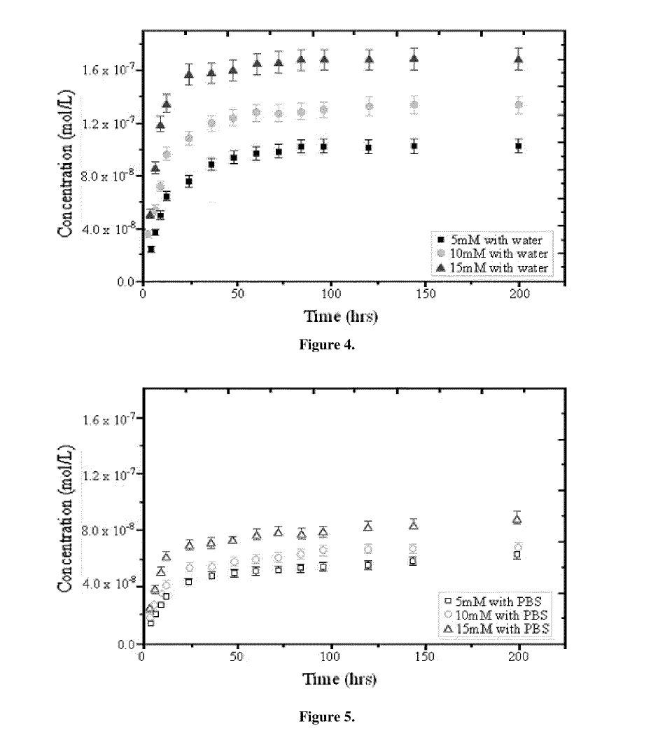

The behavior of the niosomes was next compared without a hydrogel network, when exposed to the tumor sites. An in vitro model was developed in which the niosomes were placed in dialysis tubes containing cellulose membrane. Since tumors have a slightly acidic pH (Gillies, R. J.; Liu, Z.; Bhujwalla, Z. 31P-MRS measurements of extracellular pH of tumors using 3-aminopropylphosphonate. Am Phys Cell Physiol, 1994. 267: p. C195-C203), deionized water was chosen as the in vitro medium to mimic that condition. The dialysis system was then immersed in deionized water. Samples were collected at specified time intervals and tested for its fluorescence. The niosomes initially showed exponential drug release, followed by a steady release, as indicated by the drug concentration plateaus in FIGS. 4 and 5.

After running the experiments for over a week, the niosomes with the highest concentration of the dye and the largest size, seen in FIG. 3, were seen to rupture more readily when in contact with water seen in FIG. 4. This is explained by the fact that larger niosomes have a lower surface area:volume ratio rendering them less stable. It was also observed that when water was used as the in vitro release medium, most of the dye was released within the first 24 hrs of the experiment, after which the release rate flattens out to reach steady state. The difference in the concentration of the ions between the niosomes and the medium leads to a difference in osmotic pressure resulting in an uncontrolled rupture of the niosomes. This is undesirable since the system is to deliver drugs over extended periods in a controlled manner.

To prevent the untimely rupture of the niosomes, a stable environment must be provided. The release rate of the niosomes exposed to water was tested. It was found that niosomes exposed to water possess a high release rate, seen in Table 1.

TABLE 1
Percentage of dye released when the niosomes were exposed to water.

| Time | 5 mM | 10 mM | 15 mM |
|---|---|---|---|
| (hrs) | (% released) | (% released) | (% released) |
| 0 | 0 | 0 | 0 |
| 3 | 25.97 | 30.43 | 34.82 |
| 6 | 40.14 | 46.02 | 57.55 |
| 9 | 53.40 | 59.10 | 77.79 |
| 12 | 66.76 | 77.33 | 85.83 |
| 24 | 76.72 | 84.09 | 97.50 |
| 72 | 91.75 | 95.17 | 99.79 |
| 144 | 100 | 100 | 100 |
| 200 | 100 | 100 | 100 |
| 350 | 100 | 100 | 100 |

### Example 3

### Hydrogel Preparation

Niosomes typically rupture in an uncontrolled manner and at a faster rate when exposed directly to the tumor sites due to the differences in the ionic strengths. To prevent this, a stable environment in the form of a hydrogel network must be provided, which also aid in the controlled release of the chemotherapeutic drugs. Chitosan, an amino-polysaccharide obtained by alkaline deacetylation of chitin, a natural component of shrimp or crab shells, is a biocompatible and biodegradable, pH-dependent, cationic polymer (Chenite, A; et al. Novel injectable solutions of chitosan from biodegradable gels in situ. Biomaterials, 2000: p. 2155-2161). It is a copolymer of glucosamine and N-acetyl glucosamine and is known to be digestible by lysozyme according to the amount of N-acetyl groups and their distribution in the backbone (Ruel-Gariepy, E., et al. Characterization of Thermosensitive Chitosan Gels for the Sustained Delivery of Drugs. International Journal of Pharmaceutics, 2000. 203: p. 89-98), and it is both abundant and has a low cost making it both feasible and economical. It was found that the thermo-sensitive chitosan hydrogel functioned particularly well when the degree of deacetylation is between 75-85%.

Three different molecular weights of chitosan were used to embed the niosomes encapsulated with the dye. The ranges of the molecular weights are as shown in Table 2.

TABLE 2
Range of chitosan molecular weights used to form the hydrogel.

| Low Molecular | Medium Molecular | Practical Crude |
|---|---|---|
| Weight (LMW) | Weight (MMW) | Grade (PG) |
| 50000-190000 Da | 190000-310000 Da | 190000-375000 Da |

Medium molecular weight chitosan was found to have the finest controlled release. Hence, further experiments were conducted using this grade of chitosan.

In some embodiments of the invention, the chitosan is made to respond to external stimuli such as temperature, pH and ionic strength (Molinaro, G; et al. Biocompatibility of thermosensitive chitosan-based hydrogels: an in vivo experimental approach to injectable biomaterials. Biomaterial, 2002. 23: p. 2717-2722), by the addition of certain polyols (Ta, H. T.; et al. Injectable chitosan hydrogels for localized cancer therapy. J Controlled Release, 2008. 126: p. 205-216). An exemplary compound is β-glycerophosphate, which neutralizes the chitosan solution and renders the chitosan temperature-sensitive. Such systems are liquid at room temperature and gel once the solution reaches body temperature (37° C.). This property can be especially useful in the present system, allowing the composition to be injectable and avoiding the cost of surgeries as in the case of implants. Further, the pH needed to induce gellation is varied depending on the amount of cross-linker added to it, as seen below.

β-glycerophosphate plays three essential roles; it increases the gellation pH to the physiological range of 7.0-7.4; it prevents immediate precipitation or gelation; and it allows for controlled hydrogel formation when an increase in temperature is imposed (Chenite, A; et al. Novel injectable solutions of chitosan from biodegradable gels in situ. Biomaterials, 2000: p. 2155-2161; Ruel-Gariepy, E., et al. Characterization of Thermosensitive Chitosan Gels for the Sustained Delivery of Drugs. International Journal of Pharmaceutics, 2000. 203: p. 89-98; Chenite, A; et al. Rheological characterization of thermogelling chitosa/glycerophosphate solutions. Carbohydrate Polymers, 2001. 46:p. 39-47). A range of crosslink densities were tested for their gelling and dye release rate. Table 3 shows the various ratios of the cross-linker β-Glycerophosphate and Chitosan and their corresponding pH. Gelling of the chitosan system occurred only in the ratio range (3.5):1 to (4.5):1. This corresponded to a pH range from 6.9 to 7.9 which convey the fact that the drug system is thermo-sensitive only around the physiological pH range.

TABLE 3
Crosslinking properties for differing ratios of
β-Glycerophosphate and Chitosan.

| | β-GP:Chitosan | | | | | | |
|---|---|---|---|---|---|---|---|
| | (3.0):1 | (3.25):1 | (3.5):1 | (4.0):1 | (4.5):1 | (4.75):1 | (5.0):1 |
| pH | 6.6 | 6.7 | 6.9 | 7.4 | 7.9 | 8.1 | 8.3 |

The ratio (4.0):1 gave the finest controlled release as its pH corresponded to that of the niosomes which provided greater stability to the niosomes and hence lower release. Further experiments were conducted using this crosslink density ratio.

Chitosan is typically not soluble in water, but its solutions can be obtained in acidic aqueous media which protonate chitosan amino groups, rendering the polymer positively charged and thereby overcoming associative forces between chains (Chenite, A; et al. Rheological characterization of thermogelling chitosan/glycerophosphate solutions. Carbohydrate Polymers, 2001. 46:p. 39-47). Factors which determine the ease of gelling of the chitosan is its degree of deacetylation and the molecular weight (Ruel-Gariepy, E.; et al. Thermosensitive Chitosan-Based Hydrogel Containing Lipsomes for the Delivery of Hydrophilic Molecules. J of Controlled Release, 2002. 82: p. 373-383; Ruel-Gariepy, E.; et al.; A Thermosensitive Chitosan-Based Hydrogel for the Local Delivery of Paclitaxel. Eur J of Pharm and Biopharm, 2004. 57(53-63); Ruel-Gariepy, E. and J.-C. Leroux, In Situ-Forming Hydrogels—Review of Temperature-Sensitive Systems. Eur J of Pharm and Biopharm, 2004. 58: p. 409-426; Cho, J; et al.; Physical Gelation of Chitosan in the Presence of β-Glycerophosphate: The Effect of Temperature. Biomacromolecules 2005. 6:p. 3267-3275; Kempe, S; et al.; Characterization of Thermosensitive chitosan based hydrogels by rheology and electron paramagnetic resonance spectroscopy. Eur J of Pharm and Biopharm, 2008. 68; p. 26-33; Cho, J; Heuzey, M. C. Dynamic scaling for gelation of a thermosensitive chitosan-β glycerophosphate hydrogel. Colloid Polymer Science, 2008. 286: p. 427-434; Zhou, H. Y.; et al.; Effect of molecular weight and degree of deacetylation on the preparation and characteristics of chitosan thermosensitive hydrogel as a delivery system. Carbohydrate Polymers, 2008. 73: p. 265-273; Peppas, L. B. Polymers in controlled drug delivery. Med Plastics and Biomat Magazine. 1997; Parthasarathi, G., N. Udupa, P. Umadevi, and K. Pillai, Niosome Encapsulated of Vincristine Sulfate: Improved Anticancer Activity with Reduced Toxicity in Mice. J of Drug Target, 1994. 2(2): p. 173-183).

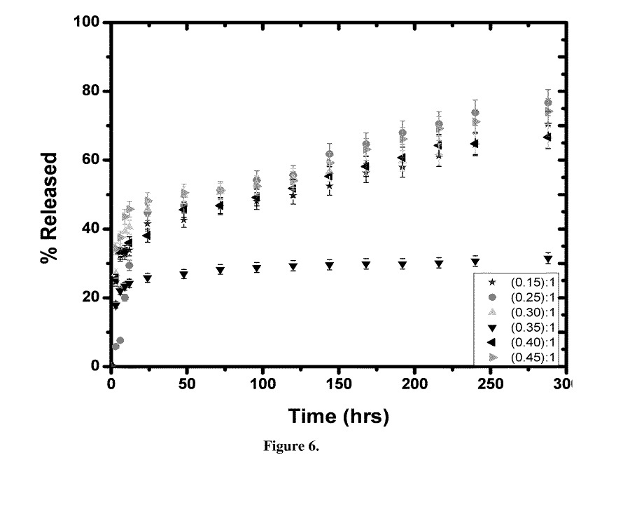

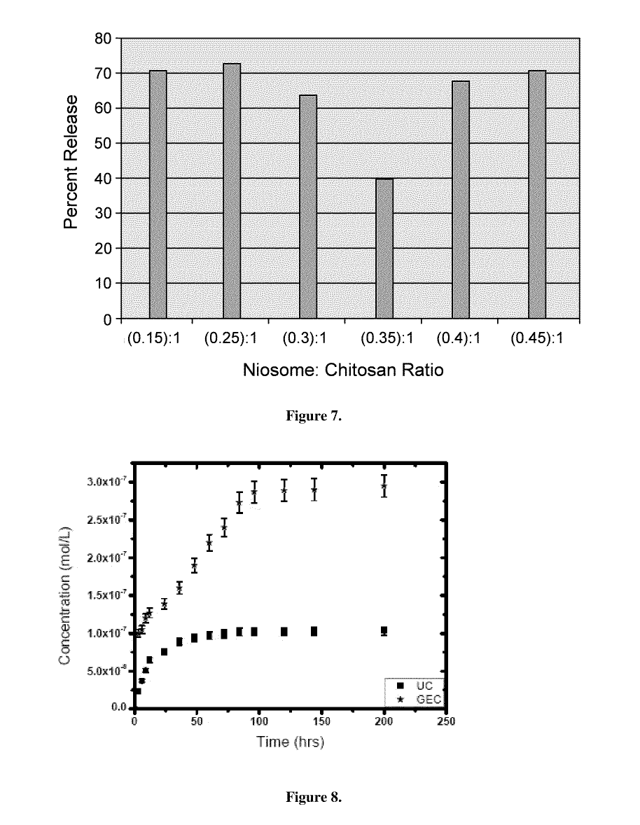

Further, the amount of niosomes loaded into the chitosan network was also found to have an effect on the rate of dye released into the surroundings. Niosome: Chitosan ratios ranging from (0.15):1 to (0.45):1 were investigated for their dye release, as seen in FIG. 6. An optimum value was obtained at a ratio of (0.35):1 which resulted in the finest controlled release. Without being bound to any theory, the optimal release is likely due to the interactions taking place between chitosan and the niosomes. It could be the result of an increase in the repulsive forces occurring between the two constituents at lower packaging densities after which it shows optimum release due to the increase in the attractive forces which results in close packing of the delivery system and hence a lower release. After the threshold level, due to the presence of more molecules repulsive forces come into play. This in turn results in a loosely packed structure and hence a higher release rate. FIG. 7 shows the final percentage released at the end of 55 days. This plot illustrates the ability to fine tune the system to obtain the required release rate by merely altering the amount of niosomes available in the crosslink network. This result is particularly important when a high dose is required for a short period of time or a low dose for an extended period. Depending on the drug dosage and time period the system can be fined tuned to suit the requirement at hand.

Other useful hydrogels may be formed using a polymer hydrogel that is adapted to respond to temperature or solution pH, such as poly(vinyl methyl ether) (PVME), poly(2-(2-methoxyethoxy)ethyl methacrylate) (PMEO2MA), acryloyl-L-proline methyl ester (A-ProOMe), poly(N,N-diethylacrylamide), poly(N-vinylcaprolactam) (PVCL), poly-(ethylene oxide) and poly(propylene oxide) block copolymer, poly(acrylamide), or poly-NIPAAm (N-isopropylacrylamide), which is described by Hoffman, et al. (U.S. Pat. No. 4,912,032); Chen, et al. (U.S. Pat. No. 6,486,213); Bae, et al. (U.S. Pat. No. 5,262,055); West, et al. (U.S. Pat. No. 6,428,811). A non-limiting example of such a poly-NIPAAm hydrogel is formed from 20% (w/v) recrystallized NIPAAm in deionized water, and methylene-bis-acrylamide (MBAAm) at a 1:750 molar ratio to NIPAAm. Ammonium persulfate (APS) and tetramethylethylenediamine (TEMED) initiated a redox reaction to polymerize the hydrogel. Previous work with these systems has shown a temperature-dependent release of drug from the system, as seen in Hoffman, et al. (U.S. Pat. No. 4,912,032).

### Example 4

### Determination of Niosomes in Cross-Linked Chitosan Polymeric Networks

During testing of the niosomes alone, it was noted that exposure of the niosomes to are exposed to the physiological environment. Tuning the pH of the chitosan to match those of the niosomes will not only prevent premature rupture but will also ensure a stable environment for the niosomes. The degradation rate of the chitosan is another factor that determines the release rate of the drugs to its final destination.

Another in vitro model was set up which mimicked the behavior of the niosomes when embedded in the hydrogel network. Phosphate Buffer Saline (PBS) was used as the medium in this case. After running the experiments for over a week, the niosomes with the highest concentration of the dye and the largest size, seen in FIG. 3, were seen to rupture more readily when in contact with water, as seen in FIG. 4. This is explained by the fact that larger niosomes have a lower surface area:volume ratio rendering them less stable. It was also observed that when water was used as the in vitro release medium, most of the dye was released within the first 24 hrs of the experiment, after which the release rate flattens out to reach steady state. The difference in the concentration of the ions between the niosomes and the medium leads to a difference in osmotic pressure resulting in an uncontrolled rupture of the niosomes. This is undesirable since the system should deliver drugs over extended periods in a controlled manner.

The release rate of the niosomes when exposed to PBS, which has a pH similar to the niosomes, was tested to identify a stable environment for the niosomes and prevent untimely niosome rupture. It was found that niosomes exposed to PBS also possess a release rate that was higher for niosomes with larger concentration of the dye, as seen in FIG. 5. However, the overall release was found to be much lower than in water-dialyzed niosomes, seen in Example 1. Further, the dye continued to be released in a controlled manner, even after 2 weeks of the experiment. Table 4 show the percentages of dye released with time for niosomes when exposed to PBS. Comparing the release rates in water and PBS, it was observed that drug release was considerably lower when exposed to PBS proving that the stability of the niosomes are maintained when exposed to an environment with similar ionic conditions.

TABLE 4
Percentage of dye released when the niosomes were exposed to PBS.

| Time | 5 mM | 10 mM | 15 mM |
|---|---|---|---|
| (hrs) | (% released) | (% released) | (% released) |
| 0 | 0 | 0 | 0 |
| 3 | 24.98 | 29.71 | 30.65 |
| 6 | 39.21 | 42.97 | 47.82 |
| 9 | 50.06 | 55.40 | 64.65 |
| 12 | 60.17 | 64.51 | 76.95 |
| 24 | 79.29 | 81.96 | 85.51 |
| 72 | 87.71 | 90.41 | 94.43 |
| 144 | 91.98 | 93.76 | 97.43 |
| 200 | 95.70 | 97.22 | 99.92 |
| 350 | 100 | 100 | 100 |

The stability and release rate of the niosomes prepared by the different methods discussed in Example 2 was undertaken. FIG. 8 shows the release rate of the niosomes using the two techniques. In the second technique, using Gel Exclusion Chromatography to separate the free dye, a higher initial release rate was observed. This can be explained by the fact that this method did not result in a good separation of the unencapsulated dye from the niosome solution. The higher initial release is due to the free dye diffusing out of the cellulose membrane. The dye's diffusion rate from niosomes was observed to fit a non-linear curve model:

$${A}_{1} = \frac{{t}_{1}}{x - {x}_{o}} ( log \frac{{y}_{0}}{y} ) ; \qquad ( 3 )$$

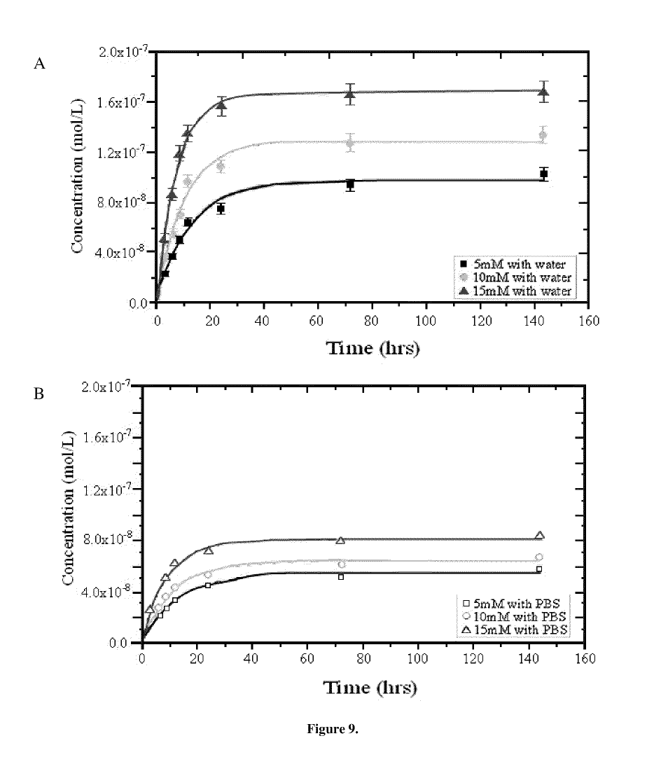

as seen in FIGS. 9(a) and (b), when water/PBS were used as the diffusion media. It was noted that this equation is similar to the equation for the diffusion coefficient using Fick' s 1st law:

$$D = ( \frac{A}{Δ t} ) log ( \frac{{C}_{0}}{{C}_{t}} ) ; \qquad ( 4 )$$

where D is the diffusion coefficient.

Niosomes were synthesized through thin film hydration and sonication, with a fluorescent dye encapsulated in the core of the niosomes. The dye, 5(6)-carboxyfluorescein, was used as a tracer dye to detect release rates and profiles during in vitro experiments. Because CF dyes have similar molecular weights to chemotherapy drugs it is a practical model for the advanced control for the release rate of drugs using nanoparticle materials.

The release rate of the CF dye was determined for the delivery system using various concentrations of dye (i.e. various niosome sizes) at various time intervals, as seen in Table 5.

TABLE 5
Values of Diffusion Coefficient for different concentrations in
water/PBS media.

| | Concentration * 10⁻³ (mol/L) | | |
|---|---|---|---|
| | 5 | 10 | 15 |
| Diffusion coefficient | 1.04E−7 | 1.39E−7 | 1.81E−7 |
| (m²/hr) in water | | | |
| Diffusion coefficient | 5.27E−8 | 6.16E−8 | 7.97E−8 |
| (m²/hr) in PBS | | | |

The concentration of the CF dye was determined using fluorescence spectrometry.

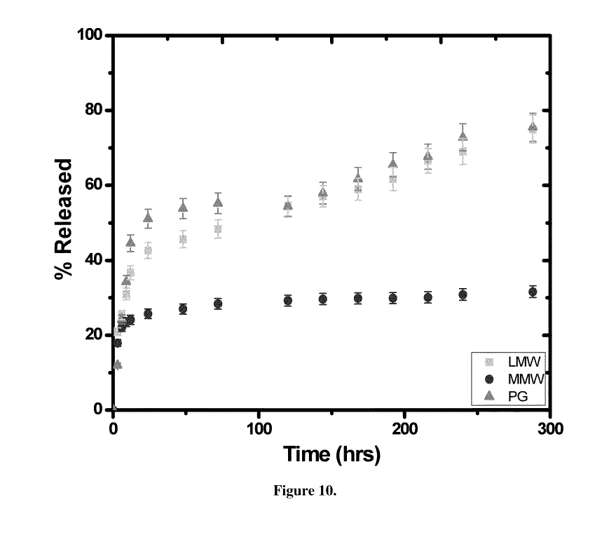

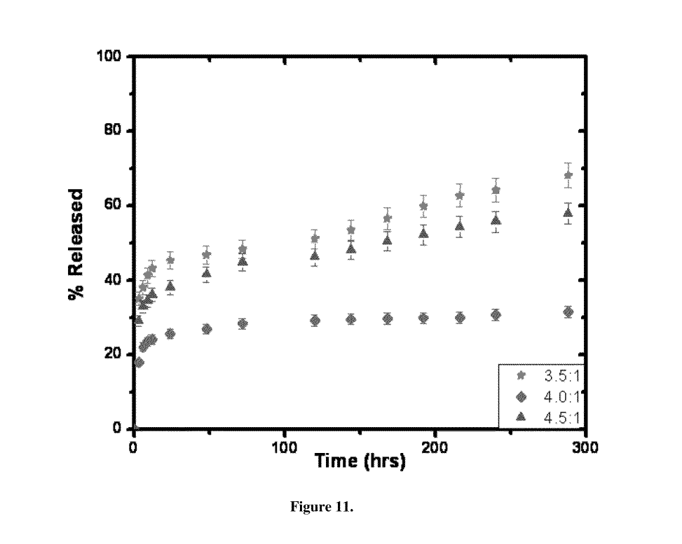

CF dye has an excitation/emission range of 492 nm to 514 nm. As indicated above, niosomes rupture uncontrollably when they are exposed to tumor-like conditions. Hence niosomes require a protective layer, such as a cross-linked chitosan network. The molecular weight of the chitosan plays a significant role in the release rate of drug/dye molecules from inside the niosome-hydrogel networks. The rate depends on the compactness or closely packed structure of the chitosan network. Three different molecular weight chitosan were used to embed the niosomes encapsulated with the dye. The ranges of the molecular weights are as shown in table 2. The medium molecular weight (MMW) chitosan was observed to have the finest controlled release as compared to the low molecular weight (LMW) chitosan, as seen in FIG. 10. An increase in molecular weight increases the chain packing, rigidity and the inter-chain bonding of the chitosan network. The result is a decrease in the swelling ratio and hence a decrease in the release rate. This leads to a more controlled diffusion of the molecules from inside the chitosan network.

The practical grade chitosan which has a molecular weight similar to the medium molecular weight chitosan was seen to have a release rate similar to the low molecular weight chitosan. This is because this grade of chitosan is not a pure grade and the impurities hinder the packed and rigid bonding between chitosan and beta-glycerphosphate. Hence relatively loose structures are formed resulting in greater release.

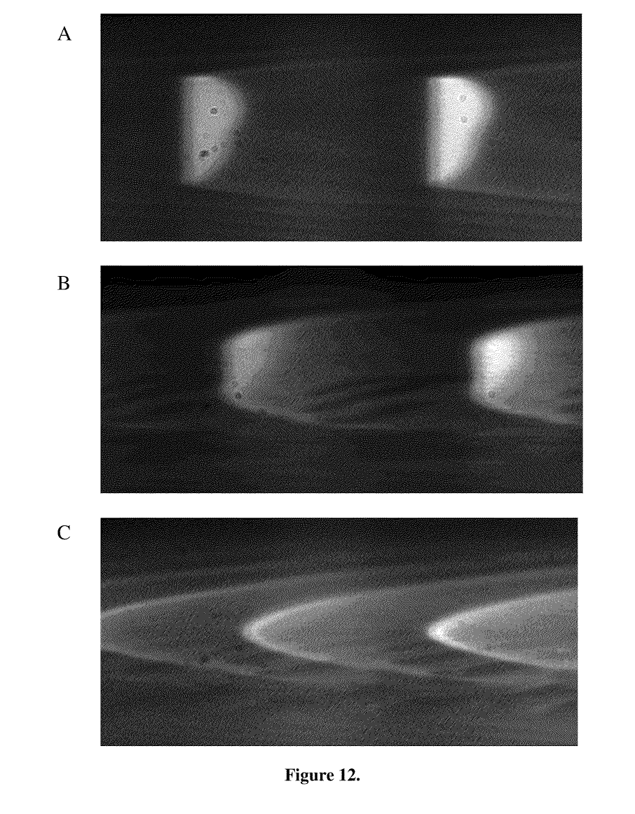

Another factor affecting the dye release rate is the amount of cross-linker available during the formation of the cross-linked network. A range of crosslink densities were tested for their gelling and dye release rate. Table 3 shows the various ratios of the crosslinker β-Glycerophosphate and Chitosan and their corresponding pH. Gelling of the system occurred only in the ratio range (3.5):1 to (4.5):1. This corresponded to a pH range from 6.9 to 7.9 which convey the fact that the system is thermo-sensitive only around the physiological pH range. FIG. 11 shows the percentage of dye released during each case. The finest controlled release was found when the pH corresponded to 7.4 which is also the pH of the niosomes used. This confirms our earlier result that niosomes will rupture at a faster rate and the release will be higher when there is a difference in the concentration of the ions in and around the niosomes. Also, the release was much higher when pH was basic as compared to the acidic pH, which establishes the fact that hydronium ions have a greater influence in the rupture of the niosome membrane than the hydroxyl ions.

### Example 5

### Immobilization of Niosomes in Cross-Linked Chitosan Polymeric Networks

The release rate of dye from niosomes embedded in chitosan-β-glycerophosphate hydrogel system prepared with different molecular weight chitosan solutions and having different degrees of deacetylation was tested. A solution of chitosan with moderate crosslinking properties using a 4:1 ratio of β-Glycerophosphate to Chitosan was studied under constant load. Surface characteristics, such as the interaction between the niosomes and chitosan, Van der Waals forces and chemical bonding were determined using Surface Forces Apparatus (SFA), seen in Table 6. The SFA plates were coated in atomically smooth mica on optically polished cylinder lenses. The data also provided information on the flexibility and response to confinement of the chitosan network to determine its packing and release of niosomes capabilities.

TABLE 6

| | Relative |
|---|---|
| Relative | hardware |
| position | units |
| 115 | 500,000 |
| 116 | 552,039 |
| 121 | 602,786 |
| 126 | 702,422 |
| 130 | 801,845 |
| 134 | 902,130 |
| 138 | 901,904 |
| 142 | 98,503 |
| 5 | 98,803 |
| 10/11 | 4,294 |
| 14 | 800,000 |
| 15 | 700,926 |
| 18 | 700,926 |
| 22 | 781,434 |
| 23 | 781,343 |
| 87 | 770,996 |
| 104 | 808,260 |
| 108 | 808,260 |
| 115 | 808,260 |
| 192 | 808,260 |
| 193 | 805,783 |
| 198 | 802,240 |
| 199 | 795,573 |
| 204 | 789,476 |
| 205 | 779,749 |
| 210 | 770,724 |
| 214 | 760,511 |
| 221 | 750,702 |
| 224 | 740,650 |
| 228 | 730,708 |
| 232 | 720,677 |
| 234 | 710,405 |
| 236 | 700,654 |
| 240 | 690,570 |
| 244 | 680,539 |
| 245 | 670,672 |
| 248 | 660,288 |
| 249 | 650,676 |
| 251 | 625,694 |
| 253 | 600,602 |
| 255 | 600,602 |
| 256 | 575,739 |
| 260 | 550,685 |
| 264-265 | 550,685 |
| 266 | 575,278 |
| 268 | 600,270 |
| 270 | 610,342 |
| 271 | 625,341 |
| 272 | 650,324 |
| 273 | 675,266 |
| 275 | 700,412 |
| 277 | 710,288 |
| 278 | 720,277 |
| 279 | 730,258 |
| 280 | 740,314 |
| 281 | 750,332 |
| 282 | 760,277 |
| 283 | 770,277 |
| 284 | 780,255 |
| 285 | 790,291 |
| 256 | 800,421 |
| 287 | 810,242 |
| 288 | 820,295 |
| 290 | 830,343 |
| 291 | 840,261 |
| 291 | 850,307 |
| 292 | 860,306 |
| 293 | 870,272 |
| 294 | 880,340 |
| 295 | 890,274 |
| 296 | 895,223 |
| 296 | 900,290 |
| 297 | 910,219 |
| 299 | 915,228 |
| 300 | 920,385 |
| 301 | 925,247 |
| 302 | 930,255 |
| 303 | 935,317 |
| 304 | 940,215 |
| 305 | 945,244 |
| 306 | 950,263 |
| 307 | 955,286 |
| 308 | 957,351 |
| 309 | 960,229 |
| 310 | 962,687 |
| 311 | 965,220 |
| 312 | 967,615 |
| 314 | 970,236 |
| 317 | 975,246 |
| 318 | 979,301 |
| 319 | 983,126 |
| 320 | 985,227 |
| 321 | 987,582 |
| 322 | 990,182 |
| 323 | 992,576 |
| 324 | 995,256 |
| 325 | 997,782 |
| 326 | 997,782 |
| 327 | 240 |
| 329 | 3,514 |
| 330 | 3,514 |
| 333 | 10,233 |
| 334 | 10,233 |
| 335-336 | 15,122 |
| 337-338 | 20,237 |
| 339 | 25,254 |
| 340 | 30,295 |
| 341 | 30,295 |
| 342 | 30,295 |
| 343 | 30,295 |
| 344 | 30,295 |
| 345 | 30,295 |
| 346 | 30,295 |
| 347 | 30,295 |
| 348 | 30,295 |
| 349 | 35,261 |
| 350 | 35,261 |
| 351 | 40,247 |
| 352 | 40,247 |
| 353 | 40,247 |
| 354 | 50,146 |
| 355 | 50,146 |
| 357 | 50,146 |
| 358 | 55,329 |
| 381 | 55,329 |
| 384 | 60,000 |

Testing the Surface Forces Apparatus experiments indicated that the hydrogel layer is very compliant and elastic. A long-range force of interaction was observed. The hydrogel is also inelastic since its elastic behavior is time-dependent, as seen in FIG. 12. The position of the fringes was observed using multiple beam interferometry, indicating that the compressed layer of chitosan prepared at under these conditions is 5.6-5.8 nm thick.

Niosomes were synthesized through thin film hydration and extrusion, with 5,(6)-carboxyfluorescein fluorescent dye encapsulated in the core of the niosomes. Niosomes were prepared using cholesterol, dicetyl phosphate (DCP), and sorbitan monosterate. The hydrogel was prepared using a glycerophosphate and chitosan thermosensitive polymer solution that begins to gel at physiological conditions of 37° C. and a pH of 6.9. The niosomes were incorporated into the hydrogel network by the use of simple physical techniques, such as mixing on-site in the brain tumor cavity. It was noted that the introduction of niosomes to the chitosan system increased the chitosan gelling rate.

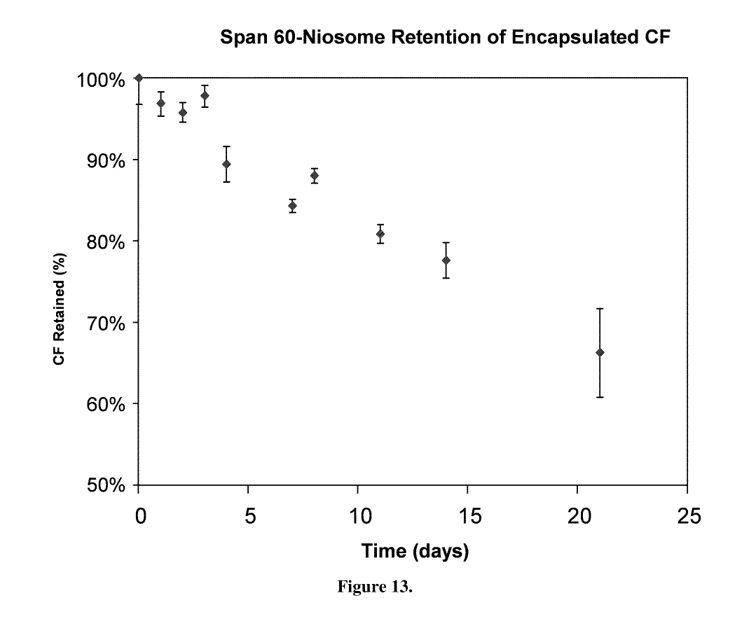

The amount of carboxyfluorescein dye that was retained in Span 60 niosomes after gel exclusion chromatography separation was then determined, as seen in FIG. 13. As can be seen, the dye is not just released randomly, but rather in a linear manner, indicating one may control for the release rate of they dye from the niosomes by manipulating other properties of the system. Table 7 is a comparison of CF encapsulation over time with varying mol % of Tween 61 included in Span 60 niosomes. It represents the amount of carboxyfluorescein dye retained in the niosomes by changing the mole percentage of Tween 61 versus time. CF concentration was monitored for 14 days for all samples between 1 and 10% Tween. Samples between 0 and 100% Tween were monitored for 9 days.

TABLE 7
Effect of Tween 61 surfactant addition on niosome drug retention.

| % Tween | % CF Retained over time |
|---|---|
| 0 | 88.0% |
| 1 | 85.9% |
| 3.25 | 84.8% |
| 5.5 | 89.1% |
| 10 | 88.0% |
| 100 | 62.2% |

Incorporation of Tween 61 into the niosomes indicates that varying the concentration of one of the niosome components can change the release of the dye. By increasing the concentration of the Tween 61, the percentage of dye that is released is increased. Similarly, other components of the niosomes, i.e. cholesterol, dicetyl phosphate, may be adjusted to optimize the release of the drug.

### Example 6

### Comparison of Niosomal Systems Using Drugs are Encapsulated in Separate Niosomes Versus Combined in One Niosome Together

The treatment for ovarian cancer includes administration of the chemotherapeutic drug Paclitaxel. Recent studies have shown that a combination of Paclitaxel and a Platinum analogue Cisplatin/Carboplatin (Armstrong, D. K., et al., Intraperitoneal cisplatin and paclitaxel in ovarian cancer. New Eng J of Med, 2006. 354(1): p. 34-43) is more effective than the traditional one drug approach. Encapsulating them in niosomes further ensures less toxicity and more control over the release.

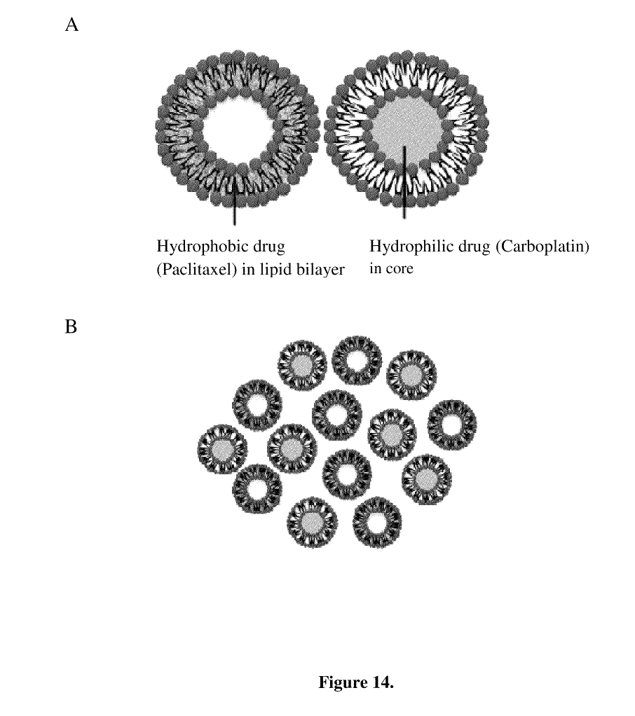

A niosomal system with each drug encapsulated in its own niosome was prepared along with a niosomal system with both drugs incorporated into the same niosomes. A thin film hydration method was used, as described above. In the first system, two different types of niosomes were prepared, seen in FIGS. 14(a) and (b). A group of hydrophobic-drug-containing niosomes were created with surfactant and the hydrophobic drug used to make the thin film and hydrated with PBS. In the hydrophilic-drug-containing niosomes, thin film was prepared with the surfactant alone and hydrated with the hydrophilic drug. The two types of niosomes were then mixed together and the stability and the release rate determined for this system too.

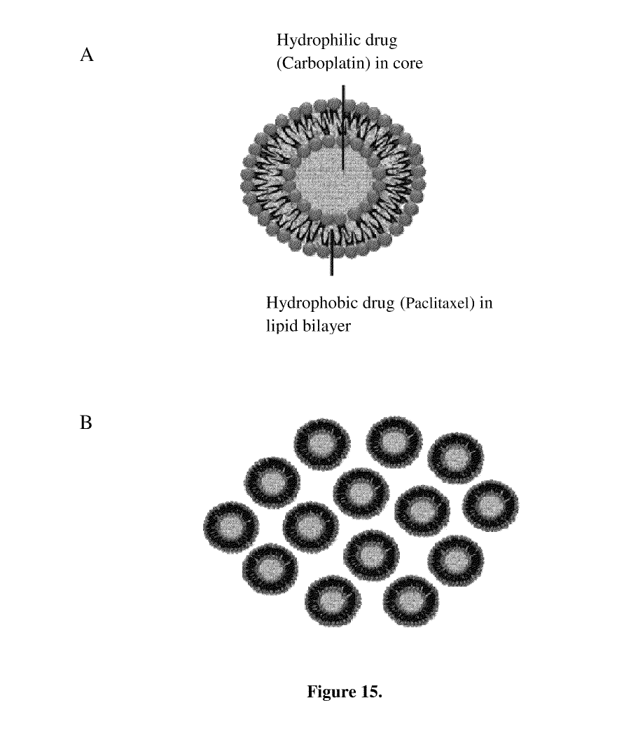

In the latter system, the thin film was prepared with the surfactant, cholesterol and the hydrophobic drug Paclitaxel. Hydration was done with the hydrophilic drug Carboplatin. This approach ensures that the hydrophobic drugs are encapsulated between the bilayers and the hydrophilic drug inside the vesicle, as seen in FIGS. 15(a) and (b). Hence the two drugs can be delivered to the targeted site at the same time in the same package.

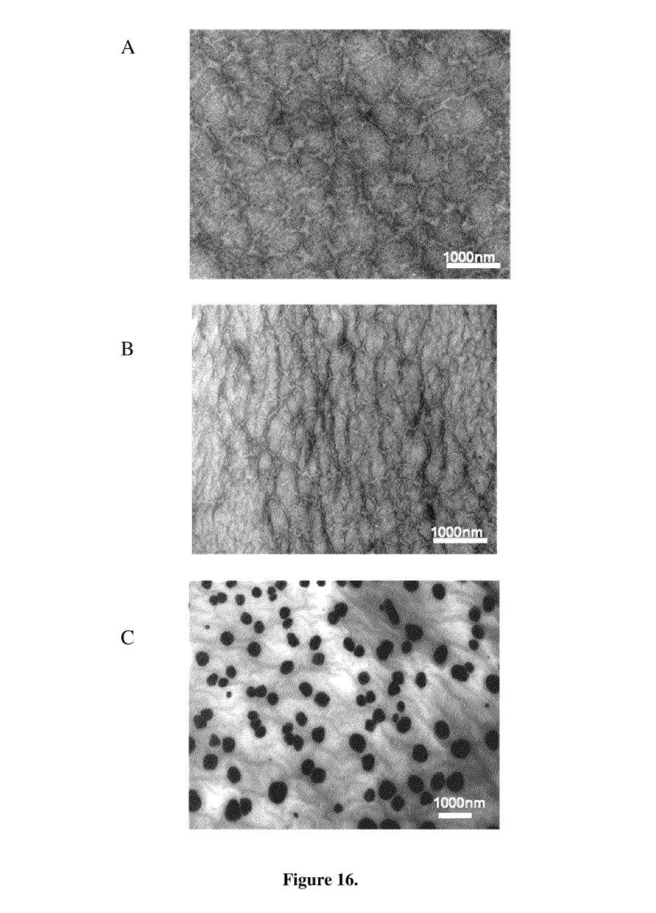

Dynamic light scattering and Transmission Electron Microscopy were used to determine the stability and aggregation, if any, taking place in the system, as seen in FIGS. 16(a)-(c). Determining the size of the niosomal system after specified periods of time gives an indication of the stability/aggregation of the niosomes. An increase in the average particle size is an indication of aggregation whereas a decrease the breakage of niosomes which indicates instability. The release rate studies were performed in vitro by exposing the chitosan-niosome system to different ionic environments. HPLC or UV-Vis spectrometer was used to determine the concentrations of the drugs diffused after each time interval. Different concentrations of the drugs were used for encapsulation. The best combination of the concentrations can be determined by one skilled in the art, taking into account which drug combinations and concentrations would be stable and amounts required of the two drugs to effect a therapeutic effect.

### Example 7

### Cytotoxicity of the Chitosan-Niosome System in Normal and Cancer Cells and Also to Determine the Selectivity of the System to Cancer Cells

To test whether chitosan-niosome system containing chemotherapeutic drugs are less toxic than when the drugs are administered directly or when encapsulated in niosomes alone, and also to study the effects of the biodegradability and biocompatibility of the delivery system, in vitro studies in cells were conducted. Normal cells and ovarian cancer cells OVCAR3 or CaOV3 (Promocell GmbH, Heidelberg, Germany; American Type Culture Collection, Rockville, Md., respectively) were obtained and cultured as described in literature. Cells were grown in Promo Cell medium or DMEM (Wisent) supplemented with 20% and 10% of FBS (Bertozzi, C. C., et al., Multiple initial culture conditions enhance the establishment of cell lines from primary ovarian cancer specimens. In Vitro Cellular & Dev Biol-Animal, 2006. 42(3-4): p. 58-62; Fernando, A., et al., Effect of culture conditions on the chemosensitivity of ovarian cancer cell lines. Anti-Cancer Drugs, 2006. 17(8): p. 913-919). Cytotoxicity studies were performed using standard MTT [3-(4,5-dimethylthiazol-2-yl)-2,5-diphenyltetrazolium bromide] dye reduction assay (Plumb, J. A., R. Milroy, and S. B. Kaye, Optimization of a Chemosensitivity Assay Based on Reduction of the Tetrazolium Dye, Mtt. Anticancer Research, 1987. 7(5): p. 902-902) on the cancer cells using the chitosan-niosome-paclitaxel system and paclitaxel alone. The number of surviving cells is directly proportional to the amount of the product formazan formed during this process. The selectivity of our system to cancer cells can be done by the following method. A mixed culture of normal cells and cancer cells were exposed to specified amounts of Chitosan-Niosome-Paclitaxel system. The uptake of drugs by the cells was then analyzed. A substantial difference in the amount of drugs inside the two kinds of cells was an indication of the specificity of the system.

As seen above, the niosomes rupture at a faster rate when they are exposed directly to tumor like conditions. Therefore it becomes essential to investigate the behavior of the niosomes by varying certain chitosan characteristics such as the molecular weight, crosslink density and the loading rate. Desired release rates were obtained by fine-tuning either the niosome or the chitosan network.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a drug delivery system, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A drug delivery medium, comprising:
at least one niosome further comprising:
a hydrophobic bilayer defining an interior hydrophilic space;
at least one hydrophobic drug integrated into the hydrophobic bilayer;
at least one hydrophilic drug encapsulated in the interior hydrophilic space; and
a polymer hydrogel, wherein the at least one niosome is embedded in the polymer hydrogel.

2. The drug delivery medium of claim 1, wherein the niosome further comprises at least one surfactant selected from the group consisting of crown ether amphiphiles bearing a steroidal moiety, 1,2-dialkyl glycerol polyoxyethylene ether, hexadecyl poly-5-oxyethylene ether, hexadecyl poly-5-oxyethylene ether (C₁₆EO₅); octadecyl poly-5-oxyethylene ether (C₁₈EO₅); hexadecyl diglycerol ether (C₁₆G₂); sorbitan monopalmitate, sorbitan monostearate, poly-24-oxyethylene cholesteryl ether, polysorbate 20, Span detergents, Brij detergents, polyoxyethylene, and polysorbates.

3. The drug delivery medium of claim 1, wherein the niosome further comprises at least one negative charged molecules selected from the group consisting of polyoxyethylene (61), sorbitan monostearate, cetyl sulphate, phosphatidic acid, phosphatidyl serine, oleic acid, palmitic acid, and dicetyl phosphates.

4. The drug delivery medium of claim 1, wherein the niosome further comprises at least one cholesterol.

5. The drug delivery medium of claim 4, wherein the niosome further comprises sorbitan monostearate, at least one cholesterol, and dicetyl phosphate at a ratio of 1:1:0.1.

6. The drug delivery medium of claim 1, wherein the polymer hydrogel is adapted to respond to temperature or solution pH.

7. The drug delivery medium of claim 6, wherein the polymer hydrogel is selected from the group consisting of chitosan, poly-NIPAAm, poly(vinyl methyl ether) (PVME), poly(2-(2-methoxyethoxy)ethyl methacrylate) (PMEO2MA), acryloyl-L-proline methyl ester (A-ProOMe), poly(N,N-diethylacrylamide), poly(N-vinylcaprolactam) (PVCL), poly-(ethylene oxide) and poly(propylene oxide) block copolymer, and poly(acrylamide).

8. The drug delivery medium of claim 7, wherein the polymer hydrogel is chitosan comprising a molecular weight within the range of 19,000 Daltons and 31,000 Daltons

9. The drug delivery medium of claim 7, wherein the polymer hydrogel further comprises β-glycerophosphate polyol, glycerol, pentaerythritol, ethylene glycol, glycerin, castor oil, sucrose polyethylene glycol, polypropylene glycol, poly(tetramethylene ether) glycol, polyoses, trehalose, glycogen, cellulose, chitin, amylose, and amylopectin glycogen.

10. The drug delivery medium of claim 9, wherein the polymer hydrogel comprises chitosan and β-glycerophosphate at a ratio of 4.0:1.

11. The drug delivery medium of claim 1, further comprising a plurality of biodegradable polymer hydrogel layers, wherein the niosomes are embedded in the hydrogel layers, and wherein the adjoining layers have distinct properties.

12. A method of preparing a drug delivery medium, comprising the steps of
combining at least one surfactant, at least one cholesterol, and at least one hydrophobic drug;
adding a solvent to the at least one surfactant, and the at least one cholesterol to form a solution
evaporating the solvent to form a thin film; and
hydrating the thin film with a hydrophilic drug.

13. The method of claim 12, further comprising agitating the solution at 60° C. until the solids dissolve.

14. The drug delivery medium of claim 12, wherein the at least one surfactant is selected from the group consisting of crown ether amphiphiles bearing a steroidal moiety, 1,2-dialkyl glycerol polyoxyethylene ether, hexadecyl poly-5-oxyethylene ether, hexadecyl poly-5-oxyethylene ether (C₁₆EO₅); octadecyl poly-5-oxyethylene ether (C₁₈EO₅); hexadecyl diglycerol ether (C₁₆G₂); sorbitan monopalmitate, sorbitan monostearate, poly-24-oxyethylene cholesteryl ether, polysorbate 20, Span detergents, Brij detergents, polyoxyethylene, and polysorbates.

15. The drug delivery medium of claim 12, wherein the niosome further comprises at least one negative charged molecules selected from the group consisting of polyoxyethylene (61), sorbitan monostearate, cetyl sulphate, phosphatidic acid, phosphatidyl serine, oleic acid, palmitic acid, and dicetyl phosphates.

16. The drug delivery medium of claim 12, wherein the solution is evaporated by passing N₂ gas over the solution.

17. The drug delivery medium of claim 12, further comprising extruding the resulting niosomes.

18. The drug delivery medium of claim 12, further comprising separating the niosomes from unincorporated hydrophilic drug by ultracentrifugation.

19. The drug delivery medium of claim 12, further comprising adding the niosomes to a hydrogel.

20. The drug delivery medium of claim 19, wherein the hydrogel is chitosan.

21. The drug delivery medium of claim 20, further comprising adding β-glycerophosphate to the chitosan solution.

22. The drug delivery medium of claim 21, wherein the β-glycerophosphate is added to the chitosan solution at a ratio of 4.0:1.

23. The drug delivery medium of claim 20, wherein the ratio of niosome to chitosan ratios ranges from (0.15):1 to (0.45):1.

24. The drug delivery medium of claim 12, wherein the noisome is composed of sorbitan monostearate, cholesterol and dicetyl phosphate combined in the ratio 1:1:(0.1).
